(19) United States
(12) Patent Application Publication
(10) Pub. No.: US 2023/0372007 A1
(43) Pub. Date: Nov. 23, 2023

(54) MEDICAL DEVICE WITH A STEERABLE TIP

(71) Applicant: Boston Scientific Medical Device Limited, Galway (IE)

(72) Inventors: Deepak Kumar Sharma, Muzaffarnagar (IN); Sharath Kumar G, Ramanagar District (IN)

(73) Assignee: Boston Scientific Medical Device Limited, Galway (IE)

(21) Appl. No.: 18/319,820

(22) Filed: May 18, 2023

Related U.S. Application Data
(60) Provisional application No. 63/343,123, filed on May 18, 2022.

(51) Int. Cl. A61B 18/14

(52) U.S. Cl. CPC A61B 18/1492; A61B 2018/00553

(57) ABSTRACT

Medical devices and methods are disclosed. An example medical device for accessing a body lumen along a biliary and/or pancreatic tract may include an elongated member having a distal end and a proximal end. The medical device may have a flexible distal tip. The flexible tip may be steerable by applying tension to one or more steering wires extending between the distal tip and a handle at a proximal end of the medical device. The medical device may be a sphincterotome.

### CROSS REFERENCE TO RELATED APPLICATIONS

This application claims the benefit of U.S. Provisional Patent Application Serial No. 63/343,123 filed on May 18, 2022, the disclosure of which is incorporated herein by reference.

### TECHNICAL FIELD

The present disclosure pertains to medical devices, and methods for manufacturing and use of these medical devices. More particularly, the present disclosure pertains to medical devices for accessing a body lumen along a biliary and/or pancreatic tract.

### BACKGROUND

A wide variety of intracorporeal medical devices have been developed for medical use, for example, for endoscopic procedures. Some of these devices include guidewires, catheters, catheter systems, endoscopic instruments, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices and medical systems.

A medical device includes an elongate tube having a proximal end and a distal end configured to be directed toward an opening of a body lumen, a wire filament extending along the elongate tube, a distal end of the wire filament connected to the distal end of the elongate tube, a proximal portion of the wire filament extending at least partially along the elongate tube, and a distal portion of the wire filament extending external to the elongate tube, a steerable distal tip extending distal of a distal end of the wire filament, and a handle in mechanical communication with the steerable distal tip. The handle is configured to be adjusted to control a position of the steerable distal tip in at least three directions.

Alternatively or additionally to any of the examples above, one or more steering wires extending from the handle to the steerable distal tip, and adjustment of the handle causes tension on one or more of the one or more steering wires to adjust the position of the steerable distal tip.

Alternatively or additionally to any of the examples above, the handle includes an actuating portion connected to the wire filament and the one or more steering wires.

Alternatively or additionally to any of the examples above, actuation of the actuating portion in a radial direction adjusts the position of the steerable distal tip.

Alternatively or additionally to any of the examples above, actuation of the actuating portion in an axial direction causes the distal end of the elongate tube to flex, and actuation of the actuating portion in a radial direction adjusts the radial position of the steerable distal tip.

Alternatively or additionally to any of the examples above, the actuating portion includes a manipulating element coupled to the one or more steering wires and configured to radially adjust about a rotational joint.

Alternatively or additionally to any of the examples above, the one or more of the steering wires extend through at least a portion of the elongate tube, and the elongate tube includes a first lumen for receiving the wire filament and one or more lumens each configured to receive one of the one or more steering wires.

Alternatively or additionally to any of the examples above, the one or more steering wires include at least three steering wires extending from the handle to the steerable distal tip.

Alternatively or additionally to any of the examples above, adjustment of the handle in a radial direction relative to the elongate tube is configured to adjust the position of the steerable distal tip.

Alternatively or additionally to any of the examples above, the handle is in communication with the proximal portion of the wire filament and adjustment of the handle in an axial direction of the elongate tube is configured to cause the distal end of the elongate tube to flex and expose a cutting portion of the wire filament.

An example system includes an endoscope having a proximal end with an axial opening, a distal end with a radial opening and a working channel extending therebetween, a guidewire, and a medical device extendable through the working channel and configured to be directed toward an opening of a body lumen comprising an elongate tube having a proximal end and a distal end, the distal end configured to extend through the radial opening, a steerable distal tip configured to extend through the radial opening, a handle in mechanical communication with the steerable distal tip, and wherein the handle is configured to be adjusted to adjust a radial position of the steerable distal tip.

Alternatively or additionally to any of the examples above, the system may include one or more steering wires extending from the handle to the steerable distal tip, and wherein adjustment of the handle causes tension on one or more of the steering wires to adjust a radial position of the steerable distal tip.

Alternatively or additionally to any of the examples above, the handle includes an actuating portion connected to the one or more steering wires, and actuation of the actuating portion in a radial direction adjusts a radial position of the steerable distal tip.

Alternatively or additionally to any of the examples above, the actuating portion includes a manipulating element coupled to the one or more steering wires and configured to radially adjust about a rotational joint.

Alternatively or additionally to any of the examples above, the medical device further comprises a wire filament extending along the elongate tube, a distal end of the wire filament is connected to the distal end of the elongate tube, a proximal portion of the wire filament extends at least partially along the elongate tube, and a distal portion of the wire filament extends external to the elongate tube.

An example method of accessing a body lumen of a subject includes extending an endoscope having a working channel into the body lumen of the subject, extending an elongate tube having a distal end and a lumen through the working channel, directing the distal end of the elongate tube through an opening of the body lumen, and steering a steerable distal tip forming a terminal end of the elongate tube toward a duct located distal of the opening of the body lumen.

Alternatively or additionally to any of the examples above, steering the steerable distal tip comprises radially adjusting a handle in communication with the steerable distal tip to steer the steerable distal tip toward the duct.

Alternatively or additionally to any of the examples above, steering the steerable distal tip comprises radially adjusting at least a portion of a handle in communication with the steerable distal tip about a rotational joint to steer the steerable distal tip toward the duct.

Alternatively or additionally to any of the examples above, the method further includes extending a guidewire through the steerable distal tip and into the duct.

Alternatively or additionally to any of the examples above, the method further includes removing the elongate tube from the body lumen and the working channel of the endoscope while maintaining the guidewire in the duct, and inserting another elongate tube over the guidewire and into the duct.

The above summary of some embodiments is not intended to describe each disclosed embodiment or every implementation of the present disclosure.

### BRIEF DESCRIPTION OF THE DRAWINGS

The disclosure may be more completely understood in consideration of the following detailed description in connection with the accompanying drawings, in which:

FIG. 1 is a schematic view of an illustrative system for accessing an opening of a body lumen of a subject;

FIG. 2 is a schematic view of the system depicted in FIG. 1, taken from circle-2;

FIG. 3 is a schematic view of an illustrative system for accessing a body lumen;

FIG. 4 is a schematic cross-sectional view of an illustrative elongate tube of an accessory medical device;

FIG. 5 is a schematic view of an illustrative system for accessing a body lumen of a subject in use to access the body lumen;

FIGS. 6-9 are schematic views of an illustrative system for accessing a body lumen of a subject being used to access the body lumen;

FIG. 10 is a schematic diagram of an illustrative method of using a system for accessing a body lumen; and

FIG. 11 is a schematic view of an illustrative handle portion of an accessory medical device.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

Although configurations of the present disclosure may be described with specific reference to medical devices and systems (e.g., endoscopic devices, accessory tools, and/or guidewires inserted through a duodenoscope, near or through a papilla, or the like) for selective access to, aligning with, and/or cannulation of the common bile duct (CBD) or pancreatic duct (PD) during an Endoscopic Retrograde Cholangiopancreatography (ERCP) procedure, it should be appreciated that such medical devices and systems may be used in a variety of medical procedures which require navigating one or more accessory tools through ductal, luminal, vascular, or body lumen anatomies, including, for example, interventional radiology procedures, balloon angioplasty/angiography procedures, thrombolysis procedures, urological or gynecological procedures, and the like. The disclosed medical devices and systems may be inserted via different access points and approaches, e.g., percutaneously, endoscopically, laparoscopically, or some combination thereof.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The detailed description is intended to illustrate but not limit the disclosure. Those skilled in the art will recognize that the various elements described may be arranged in various combinations and configurations without departing from the scope of the disclosure. The detailed description illustrates example embodiments of the disclosure.

As discussed herein, it may be desirable for a distal tip of a medical device to be flexible to navigate effectively through a body lumen. For example, flexible distal tips of sphincterotomes may be capable of facilitating navigation through narrow passages such as the papilla of Vater, the common bile duct, the pancreatic duct, and/or other passages. In some instances, a flexible distal tip of a sphincterotome or other suitable medical device may facilitate steering the sphincterotome into a target body lumen that is closely situated to structures such as lesions, stones or other build-up and/or has such structures situated therein.

In some instances, the devices and methods that are disclosed herein may be useful for diagnostic or therapeutic procedures in the biliary and/or pancreatic tracts, along with being useful for other purposes. Access to the pancreaticobiliary system, as facilitated by the devices disclosed herein, may be required to diagnose and/or treat a variety of conditions, including but not limited to tumors, gallstones, infections, sclerosis, and pseudo cysts. The devices disclosed herein may also be useful for navigation in other parts of the body such as the cardiovascular system and so forth.

Endoscopic retrograde cholangiopancreatography (ERCP) may be used to diagnose and treat conditions of the common bile duct, including, for example, gallstones, inflammatory strictures, leaks (e.g., from trauma, surgery, etc.), and cancer. In an ERCP procedures, a physician may view, through an endoscope, the inside of the stomach and/or the duodenum. Often, dyes may be injected (e.g., via a lumen of a sphincterotome or other device) into the ducts in the biliary tree and pancreas so that the area can be seen using X-rays. These procedures may necessitate gaining and keeping access to the papilla of Vater, the common bile duct, and/or the pancreatic duct, which may be technically challenging, may require extensive training and practice to gain proficiency, and may require one or more expensive tools in order to perform.

During an ERCP procedure, a number of steps are typically performed while the patient is often sedated and/or anaesthetized. For example, an endoscope or duodenoscope may be inserted through the mouth, down the esophagus, into the stomach, through the pylorus into the duodenum, to a position at or near the papilla of Vater (also referred to as the ampulla of Vater), which is the opening of the common bile duct and the pancreatic duct. Due to the shape of the papilla, and the angle at which the common bile and pancreatic ducts meet the wall of the duodenum, the distal end of the endoscope or duodenoscope is generally placed just past the papilla. Due to this positioning beyond the papilla, the endoscopes or duodenoscopes typically used in these procedures are usually side-viewing devices. The side-viewing feature provides imaging along the lateral aspect of the tip rather than from the end of the endoscope. Such orientation may allow a clinician to obtain an image of the medial wall of the duodenum, where the papilla of Vater is located, even though the distal tip of the viewing device is beyond the opening. Once the papilla or a target area in the duodenum is visually located, an accessory medical device, such as sphincterotome or other accessory medical device, may be extended out a side opening or window of the endoscope or duodenoscope for facilitating access through the papilla and into a desired one of the common bile duct and the pancreatic duct.

Although not required, a fluid (e.g., a compressible fluid, such as compressible gas, air, nitrogen, carbon dioxide, etc., and/or other suitable fluid) may be delivered via catheter (e.g., as inserted through a subject as discussed above or otherwise inserted), a sphincterotome, and/or other elongated tubular device extending through an inserted endoscope, and/or other suitable elongate medical device to an opening (e.g., the papilla of Vater) of a body lumen of a subject. Applying the fluid to the opening may facilitate identifying and/or accessing the body lumen by at least partially dilating the opening with the fluid prior to and/or during delivery of a catheter, sphincterotome, guidewire, etc. to a target anatomical structure or tissue (e.g., a duct or other suitable structure or tissue) at or distal of the opening of the body lumen. Some example techniques and systems for cannulating an opening of a body lumen using fluid are described in U.S. Patent Application Publication No. 2019/0380565, which was filed on Jun. 13, 2019, and titled DEVICES, SYSTEMS AND METHOD FOR ACCESSING A BODY LUMEN, which is hereby incorporated by reference for any and all purposes.

Alternatively or in addition to using fluid to open the opening of the body lumen, the opening may be manually opened by engaging a distal end or tip of an accessory medical device with the papilla. Further, the accessory medical device may be manipulated and/or the endoscope through which the accessory medical device extends may be manipulated to direct the accessory medical device through the papilla and into a desired one of the common bile duct and the pancreatic duct. Some example techniques and systems for manipulating a distal tip of a medical device, such as a guidewire, to access a desired body lumen are described in U.S. Patent Application Publication No. 2015/0073391, which was filed on Sep. 11, 2014, and titled MEDICAL DEVICE WITH A MOVABLE TIP, which is hereby incorporated by reference for any and all purposes.

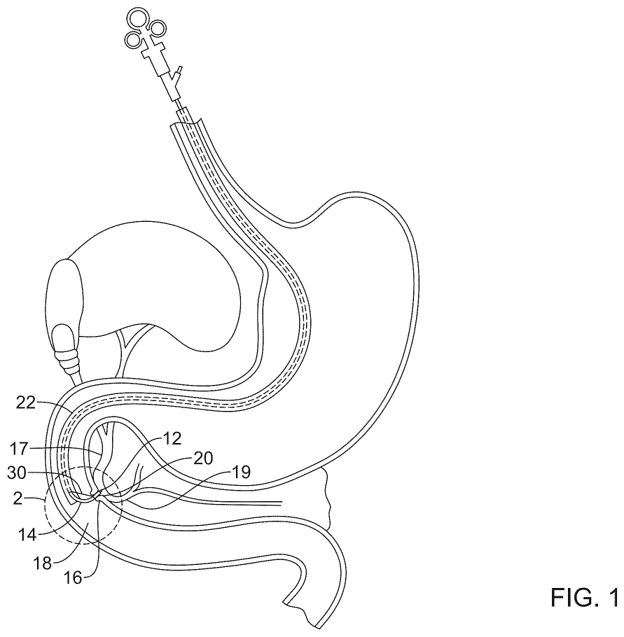

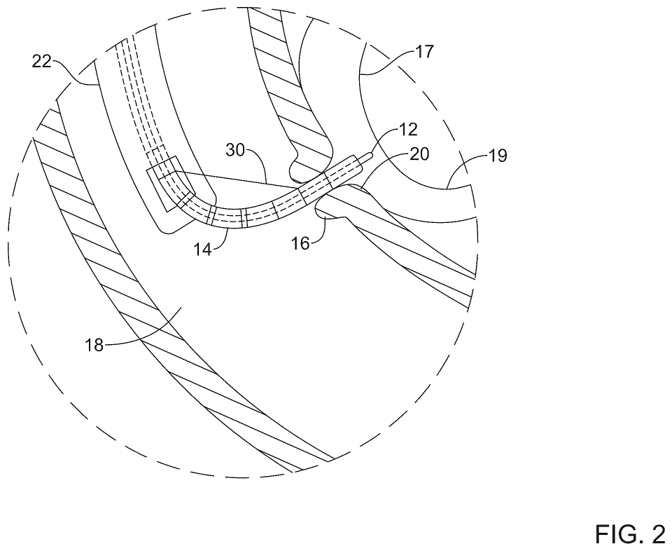

Turning to the Figures, FIGS. 1 and 2 depict a selective cannulation during an ERCP procedure, which includes a guidewire 12 and/or an accessory medical device 14 (e.g., an endoscopic accessory device, such as a sphincterotome, and/or other suitable accessory medical device) being passed towards, against, and/or through a body lumen such as the major papilla 16 (e.g., ampullary entry) near the descending duodenum 18 to access the Sphincter of Oddi Complex 20. During the cannulation procedure, a distal portion of a medical device 22 (e.g., an endoscope, duodenoscope, guide catheter, etc.) may be positioned within the descending duodenum 18. The guidewire 12 and the accessory medical device 14 may be advanced through a working channel (e.g., a lumen) of the medical device 22 towards the major papilla 16. Additionally, the guidewire 12 and/or the accessory medical device 14 may be advanced against and/or through the major papilla 16 to one of the common bile duct 17 and the pancreatic duct 19.

Accessing the papilla 16, the common bile duct 17, and/or the pancreatic duct 19 may be difficult because the openings are small compared to a size (e.g., a diameter or other size measurement) of many accessory medical devices, the openings may be closely located, the openings may be completely collapsed/closed, the openings may extend into the descending duodenum 18 at an angle that may be difficult to visualize and/or access, and/or it may be difficult to control a position of a distal tip of the accessory medical device 14. Thus, a medical professional may be required to manipulate the accessory medical device 14 and guidewire 12 by manually rotating the medical device 22, pulling on a wire filament 30 to adjust the accessory medical device 14 in a single direction, and/or use an elevator and/or other suitable ramped surface within the distal end of the medical device 22 in an attempt to better align or orient the accessory medical device 14 and/or the guidewire 12 with respect to the medical device 22 and the openings of the papilla 16, the common bile duct 17, and/or the pancreatic duct 19. Difficult cannulation procedures in which the medical professional fails to access the body lumen within a certain time limit or after a certain number of unsuccessful attempts may lead to significant post-procedure complications, such as post-ERCP pancreatitis (PEP).

A medical professional accessing a body lumen (e.g., a duct, a papilla, a common bile duct, a pancreatic duct, or the like) by manipulating an accessory medical device against or into the opening of the body lumen, as discussed above, may subject the walls of the opening of the body lumen to compressive forces. Compression of a body lumen opening or the body lumen itself can cause buckling in what may be described as the “accordion effect”. As such, it is desirable to have control over a distal tip of the accessory medical device 14 such that a user may precisely access an opening of the papilla 16 and once through the papilla 16, direct the accessory medical device 14 and/or guidewire 12 to a desired one of the common bile duct 17 and the pancreatic duct 19.

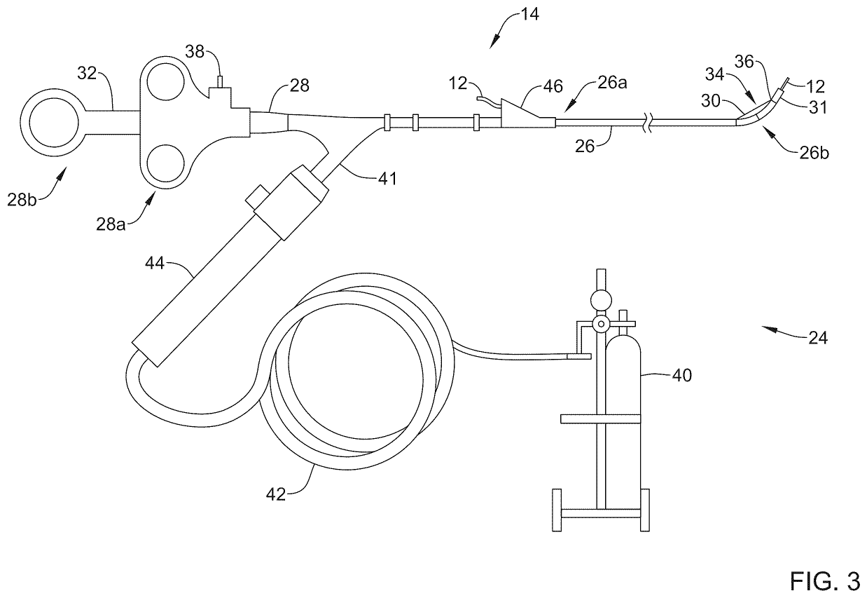

FIG. 3 schematically depicts an illustrative system 24 for accessing a body lumen. In some cases, the system 24 may include, among other components, the accessory medical device 14 with a flexible elongate tube 26. The tube 26 may have a proximal end 26a and a distal end 26b. The distal end 26b of the tube 26 may have a distal tip 31 extending distal of a distal end 36 of the wire filament 30 and may be configured to be directed toward and/or through an opening of a body lumen. The proximal end 26a of the tube 26 may be connected to, coupled with, or otherwise in communication with a handle 28.

The handle 28 may be any suitable type of handle. In one example, the handle 28may have a first portion 28a configured to receive fingers (e.g., receive two fingers) of a user using the accessory medical device 14 to hold the accessory medical device 14 and a second portion 28b configured to receive a thumb of the user. In some cases, the second portion 28b of the handle 28 may be an actuating portion 32 of the handle and a user may adjust the second portion 28b relative to the first portion 28a to control positioning of a distal end of the accessory medical device 14, as discussed herein or otherwise.

As schematically depicted in FIG. 3, the first portion 28a of the handle 28 may have two finger holes configured to receive two fingers from the user and the second portion 28b may have a thumb hole configured to receive a thumb form the user, but other configurations of the first and second portions 28a, 28b of the handle 28 are contemplated. Although not required, the handle 28, as illustrated, may be held and/or operated with a single hand of a user.

The second portion 28b of the handle 28 may be configured to be adjusted or manipulated to control a position of a distal end 26b of the elongate tube 26 and/or the wire filament 30. In some cases, the second portion 28b may be configured to adjust axially or longitudinally and radially or rotationally to adjust a position of the distal end 26b of the elongated tube 26 and/or the wire 30 in a plurality of directions.

A wire filament 30 may extend along the elongate tube 26 and may be energized for the purpose of cutting tissue within a subject. The wire filament 30 may have a proximal end (not shown) connected to an actuating portion 32 (e.g., the second portion 28b) of the handle 28, and a proximal portion extending at least partially within the tube 26. The wire filament 30 also may have a distal portion 34 (e.g., a cutting portion) extending external to the elongate tube 26, and a distal end 36 of the filament 30 may be connected to the distal end 26b of the elongate tube 26.

The wire filament 30 may be received within a lumen of the tube 26 at least along a proximal portion of the filament 30, and may be configured to slide within the lumen. In some cases, the actuating portion 32 of the handle 28 may be coupled to the wire filament 30 and manipulated to slide the wire filament 30 along the tube 26 (e.g., within the lumen of the tube 26). In one example, the actuating portion 32 (e.g., the second portion 28b) of the handle 28 may be longitudinally adjusted relative to the first portion 28a of the handle 28 to apply and remove tension to and/or from the wire filament 30.

Sliding or adjusting the wire filament 30 may result in the distal end 26b of the elongate tube 26 moving responsively to the sliding of the wire filament 30. As such, the actuating portion 32 of the handle 28 may be manipulated to control or adjust a position of the distal end 26b of the elongate tube 26, for example, to direct the distal end 26b toward the opening of a body lumen. In some cases, natural tension in or acting on the tube 26 and/or the wire filament 30 may result in the wire filament 30 returning to a relaxed position or state once a tension applied to the wire filament 30 via the handle 28 is released.

In some cases, the wire filament 30 may be utilized to electrically cut or remove tissue of the subject (e.g., to cut and cauterize tissue) and may be considered a cautery wire. As such, the handle 28 or other suitable portion of the medical device 22 may include an electrical connection 38 for an energy source (e.g., a radiofrequency energy source, or the like) to energize the wire filament 30 and facilitate cutting tissue. When the actuating portion 32 of the handle 28 is manipulated to adjust a position of the distal end 26b of the tube 26 and/or utilize the wire filament 30 for cutting tissue, the wire filament 30 may be spaced from the tube 26 and electrified to facilitate cutting tissue at or adjacent to the opening of the body lumen.

The distal tip 31 of the elongate tube may be flexible and configured to adjust in response to steering with or adjustment of a control configuration at a proximal end of the accessory medical device 14. Although the distal end 26b of the elongate tube 26 of the accessory medical device 14 may be configured to bend or otherwise adjust in response to actuation of the wire filament 30 or in response to being advanced along another device (e.g., an elevator of a duodenoscope and/or other suitable device) that directs a position of the elongated tube 26, such control over or manipulation of the distal end 26b of the elongate tube 26 using the wire filament 30 may be limited to adjustment in the direction of the wire filament 30 or in the direction provided by the distal end 26b being advanced along the other device and lack precise control needed to access a desired body lumen (e.g., a common bile duct 17, a pancreatic duct 19, etc.) To facilitate precisely controlling a distal end of the accessory medical device 14 while the device 14 is within a subject, the distal tip 31 may be configured to be adjustable or steerable in two or more directions (e.g., at least three directions, at least four directions, any desired direction, etc.) through control at the proximal end of the accessory medical device 14 such that distal end 26b of the elongate tube 26 may be guided to a desired body lumen of a subject using finer manipulation of the distal tip 31 than is available via manipulation of the elongate tube using the wire filament 30 and/or interactions with other medical devices. The distal tip 31 may be a steerable distal tip.

The distal tip 31 may be formed from any suitable material configured to bend, deflect, or otherwise move in response to forces acting thereon. In one example, the distal tip may be formed from a polyether block amide (PEBA, such as, for example, PEBAX®), but this is not required and other suitable materials are contemplated.

A positioning of the distal tip 31 may be controlled or adjusted in any suitable manner. In some cases, a plurality of wires (e.g., steering wires), such as two wires, three wires, four wires, or other suitable number of wires, may be utilized to facilitate control of the distal tip 31. Three or four wires may be utilized to provide adequate, precise control over the distal tip 31 to facilitate entry into a desired body lumen, while still fitting within existing configurations of sphincterotomes used in ERCP procedures and/or other suitable accessory medical devices 14. In one example, four wires may be utilized to facilitate control of a position of the distal tip 31.

The wires used to control the distal tip 31 may have a distal end coupled to the distal tip 31, may extend through one or more lumens of the elongate tube 26 to the handle 28, and may have a proximal end coupled to the actuating portion 32 (e.g., the second portion 28b) of the handle 28. Although two or more of the wires may extend through a single lumen of the elongate tube 26, in one example configuration, each of the wires may extend through an individual lumen (e.g., a single lumen may be provided in the accessory medical device 14 for each wire).

A distal end of the wires may be coupled to the distal tip 31 and/or a proximal end of the wires may be coupled to the actuating portion 32 of the handle 28 in any suitable manner. In one example, the wires may be coupled to the distal tip 31 and/or the actuating portion 32 of the handle 28 using adhesion, for example, through ultraviolet (UV) curing techniques, crimping techniques, and/or other suitable connecting or coupling techniques.

In addition to the one or more lumens in which the wires for controlling a position of the distal tip 31, when included, extend, the accessory medical device 14 may include one or more other lumens extending from at least the proximal end 26a of the tube 26 to the distal end 26b of the tube 26. In some cases, one or more of the lumens may have a distal opening through the distal tip 31 of the elongate tube 26.

In some cases, a lumen of the one or more lumens of the elongate tube 26 may have a fluid connection at a proximal end for a fluid source (e.g., a pressurized fluid source 40 such as a CO₂ tank, a contrast fluid source such as a syringe, etc.) The lumen may be configured to apply a fluid from the fluid source through an opening at the distal end 26b of the elongated tube 26 to a body lumen of the subject.

Although FIG. 3 depicts the fluid source 40 coupled to a fluid port 41 of the accessory medical device 14 via a fluid tube 42 and a flow regulator 44, the fluid source and/or other suitable fluid sources (e.g., syringes, etc.) may be coupled to the fluid port 41in other suitable manners. In one example, a syringe with contrast fluid may be coupled to the fluid port 41 via a luer lock connection and/or other suitable connection. Contrast agent may be delivered into the body lumen and may be used to temporarily improve imaging of the inside of the body lumen by, for example, x-ray, computed tomography (CT), or magnetic resonance (MR) imaging, ultrasound, and the like. Alternatively, separate lumens and separate fluid ports may be provided for each of a pressurized fluid and a contrast fluid.

A further lumen of the one or more lumens may be configured to accept the guidewire 12 and/or one or more other suitable medical devices usable with the accessory medical device 14. The further lumen may extend from the distal end 26b of the tube 26 (e.g., an opening at the distal tip 31) at least partially along the tube 26 toward the proximal end 26a of the tube 26.

In some cases, the guidewire 12 or other suitable medical devices may extend through the further lumen of the elongate tube 26 and proximally out of an aperture port 46. The aperture port 46 may be located at the distal end of the handle 28 and/or at one or more other suitable locations. In some cases, the aperture port 46 and the tube 26 may be configured so that the guidewire 12 and/or other suitable medical devices may be stripped through the side of the tube 26 for a rapid removal or exchange of devices, but this is not required.

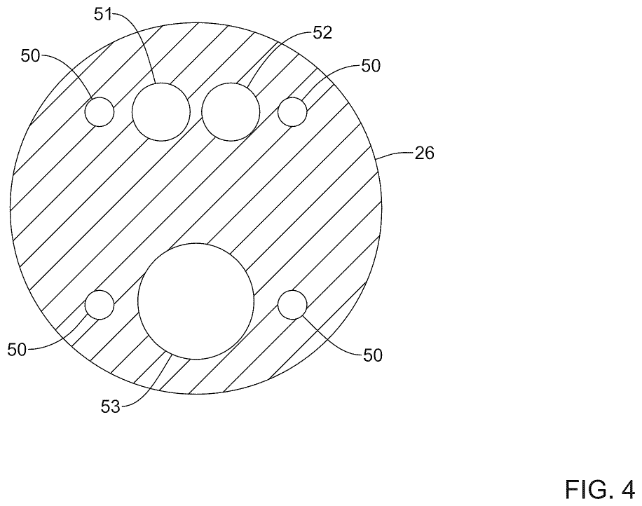

FIG. 4 schematically depicts a cross-sectional view of the elongate tube 26 depicting one example of the one or more lumens extending through the elongate tube 26, taken at a location distal of the aperture port 46 and proximal of the distal end of the distal tip 31. Although other configurations are contemplated, the elongate tube 26 may include a plurality of steering wire lumens 50 (e.g., four steering wire lumens 50, as shown in FIG. 4, and/or other suitable number of steering wire lumens 50), one or more fluid lumens 51, one or more wire filament lumens 52, and one or more medical device lumens 53. As depicted in FIG. 4, the elongate tube 26 may include four steering wire lumens 50, where each steering wire lumen is for an individual wire, a fluid lumen 51, where the fluid lumen 51 may be for pressurized fluid and/or for contrast fluid, a wire filament lumen 52 for the wire filament 30 connectable to the electrical connection 38, and a medical device lumen 53 for receiving a guidewire or other suitable medical device.

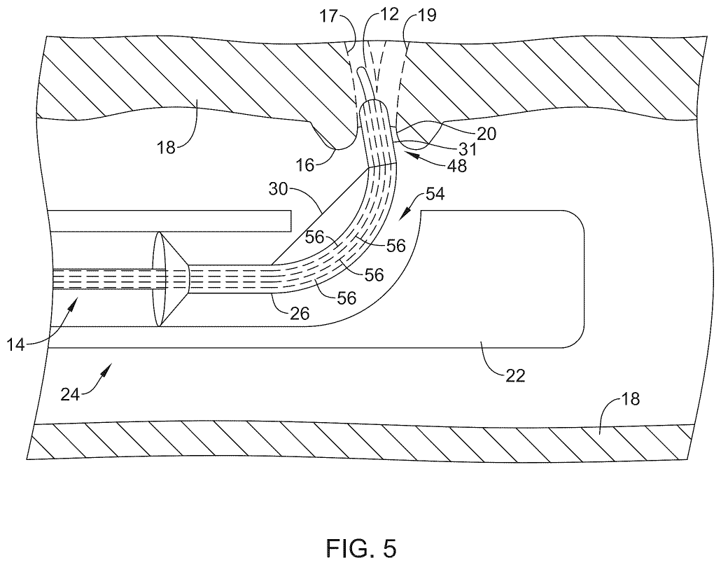

FIG. 5 depicts an enlarged distal end of the system 24 with the medial device 22 positioned adjacent an opening 48 (e.g., an opening defined by the major papilla 16) of a body lumen (e.g., a body lumen defined by the sphincter of Oddi 20). As depicted in FIG. 5, tension has been applied to the wire filament 30 to cause the distal end 26b of the elongate tube 26 to bend in the direction of the wire filament 30 exiting the medical device 22 through an opening 54 thereof. As the distal tip 31 enters through the opening 48 and into a body lumen of the subject, a position of the distal tip 31 of the elongate tube 26 may be adjusted by manipulating (e.g., selectively tensioning or otherwise applying tension to) steering wires 56 and directed to enter the common bile duct 17 as the accessory medical device 14 extends through the medical device 22. However, the steering wires 56 may be manipulated in one or more other suitable manners to direct the distal tip 31 to enter one or more other suitable body lumens (e.g., the pancreatic duct 19, etc.)

In some cases, the steering wires 56 may extend from the distal tip 31 to the handle 28 (e.g., to the actuating portion 32 of the handle 28 and/or other suitable portion of the handles). When so configured, the handle 28 may be adjusted or manipulated to cause tension on one or more of the steering wires 56 to adjust a position of the distal tip 31.

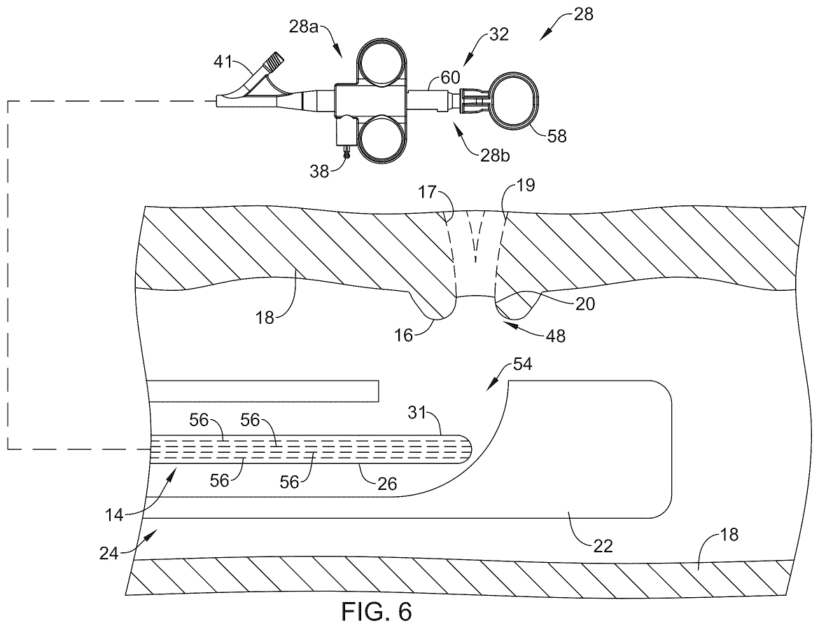

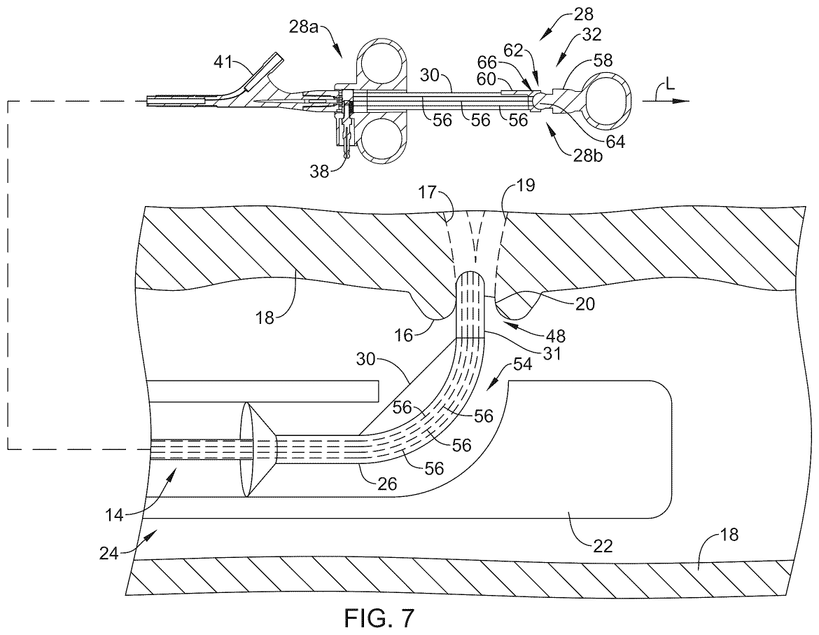

FIGS. 6-9 schematically depict positions of components of the system 24 when controlling the distal end 26b of the accessory medical device 14 by adjustments made at the handle 28 to apply tension to the wire filament 30 and/or the steering wires 56. In FIG. 6, the handle 28 is schematically depicted from a side perspective view. In FIGS. 7-9, the handle 28 is schematically depicted in cross-sectional views to facilitate depicting adjustments or movements of the actuating portion 32 that result in adjusting the distal end 26b of the elongate tube 26.

As depicted in FIG. 6, the system 24 is positioned within a duodenum 18 of a subject and the opening 54 of the medial device 22 has been positioned proximate the papilla of Vater 16 of the subject near (e.g., slightly distal of) the opening 48 of the body lumen. Initially, the distal end 26b of the elongate tube 26 may be positioned within the medical device 22 proximate the papilla 16 with the second portion 28b of the handle 28 (e.g., the actuating portion 32, which is referred to as the second portion 28b of the handle 28 with respect to FIGS. 6-9) in a resting or relaxed position relative to the first portion 28a thereof.

FIG. 7 schematically depicts a tension applied on the wire filament 30 by longitudinally adjusting, in a direction of arrow L, the second portion 28b (e.g., the actuating portion 32) of the handle 28 relative to the first portion 28a, which may cause the distal end 26b of the elongate tube 26 to bend in a direction of the wire filament 30. Prior, while, or after tension is applied to the wire filament 30, the accessory medical device 14 may be advanced in a distal direction relative to the medical device 22, such that a distal end 26b of the elongate tube 26 may exit the opening 54 of the medical device 22 and the distal tip 31 may approach and/or enter the opening 48 at the papilla 16.

As depicted in FIG. 7, a proximal end of the wire filament 30 may be coupled to the stem portion 60 and steering wires 56 may be coupled to a joint 62 of the second portion 28b of the handle 28. Further, although the configuration of the system 24 depicted in FIGS. 6-9 may include four steering wires 56, the schematic cross-sectional views of the handle 28 in FIGS. 7-9 depict only three of the four steering wires 56 as one of the steering wires 56 may extend through an omitted portion of the handle 28.

As can be seen in the cross-sectional view of the handle 28 in FIG. 7, a manipulating portion 58 of the second portion 28b of the handle 28 may be coupled to a stem portion 60 of the second portion 28b via the joint 62 (e.g., a rotational joint or other suitable type of joint). When the second portion 28b of the handle 28 is longitudinally adjusted in the direction of arrow L, the joint 62 may facilitate longitudinal adjustment of the manipulating portion 58 and the stem portion 60 together relative to the first portion 28a of the handle 28.

Once the distal tip 31 has entered the opening 48 and/or at one or more other suitable times, the wire filament 30 may be utilized to cut or otherwise remove tissue from around the opening 48. In one example, energy may be applied to the wire filament and the electrified wire filament 30 may be brought into contact with the tissue around the opening 48 by rotating the accessory medical device 14. Other techniques may be utilized to remove tissue around the opening 48 using the wire filament 30.

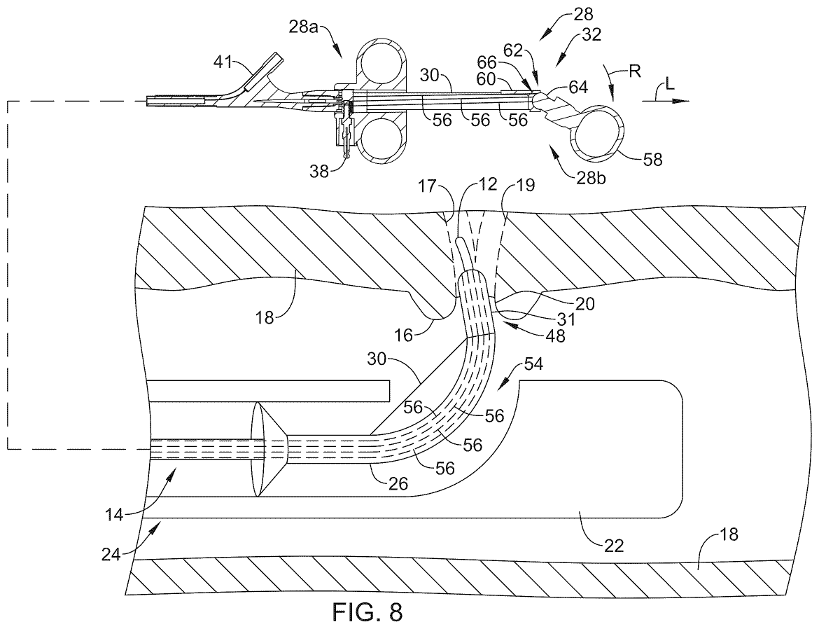

FIG. 8 schematically depicts the accessory medical device 14 with a tension on the wire filament 30 and a manipulating portion 58 of the second portion 28b (e.g., the actuating portion 32) of the handle 28 that is coupled to the steering wires 56, where the manipulating portion 58 may be rotated or manipulated in a radial direction of arrow R with respect to a longitudinal axis of the handle 28 to apply a tension to one or more steering wires 56. Such rotation and/or radial positioning of the manipulation portion 58 and the associated tension on the steering wires 56 may result in movement of the distal tip 31, as depicted in FIG. 8, to direct the guidewire 12 and the elongate tube 26 into the common bile duct 17. Although the manipulating portion 58 of the second portion 28b of the handle 28 has been depicted as causing the distal tip 31 to adjust toward the common bile duct 17, a user may manipulate the manipulating portion 58 in one or more other suitable manners to direct the distal tip 31 in one or more other directions (e.g., toward the pancreatic duct 19 and/or other suitable ducts). Prior to, while, or after the distal tip 31 is directed toward the common bile duct 17 and/or the manipulating portion 58 is manipulated, the accessory medical device 14 and/or the guidewire 12 may be advanced in a distal direction relative to the medical device 22, such the distal tip 31 and/or the guidewire 12 may approach and/or enter the common bile duct 17.

In some cases, the joint 62 coupling the manipulating portion 58 and the stem 60 may facilitate rotational and/or radial movement of the manipulating portion 58 relative to the stem portion 60, along with the longitudinal movement of the manipulating portion 58 with the stem portion 60 relative to the first portion 28a of the handle 28. Although other joint configurations are contemplated, in one example, the joint 62 between the manipulating portion 58 and the stem portion 60 of the second portion 28b of the handle 28 may be ball-socket joint. As depicted in FIG. 8, the ball 64 of the ball-socket joint may be part of the manipulating portion 58 and in communication with the steering wires 56s, while the socket 66 may be part of the stem portion 60, but this is not required and other configurations are contemplated.

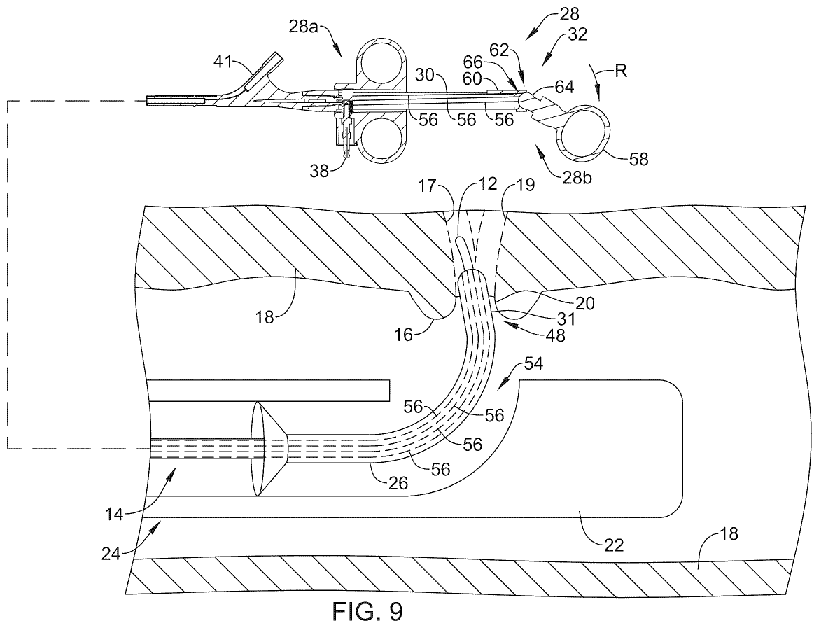

FIG. 9 schematically depicts the accessory medical device 14 with the manipulating portion 58 of the second portion 28b of the handle 28 rotated or manipulated in a radial direction of the arrow R with respect to a longitudinal axis of the handle 28. Further, relative to FIGS. 7 and 8, the longitudinal force on the second portion 28b of the handle 28 in the direction of arrow L has been released and the wire filament has returned to a relaxed state. As a result of the configuration of the second portion 28b of the handle 28, the wire filament 30 may no longer be tensioned and in a cutting position, spaced away from the elongated tube 26, but the distal tip 31 may directed toward the common bile duct 17 due to radial adjustment of the manipulating portion 58 relative to the stem portion 60 of the second portion 28b of the handle 28 in the direction of the arrow R with respect to a longitudinal axis of the handle 28.

In some cases, it may be advantageous to control or adjust the distal tip 31 of the elongate tube 26 individually or separately from the rest of the distal portion 26b of the elongate tube 26. In one example, fine adjustment of the distal tip 31 using the manipulating portion 58 of the handle 28, as discussed herein or otherwise, may allow for directing the accessory medical device 14 and/or the guidewire 12 therein to a body lumen of a subject that is in a direction different than a bend in the elongate tube caused by tensioning the wire filament 30 and/or may facilitate directing or positioning the elongate tube 26 or the guidewire 12 extending therein toward a target body lumen after tension has been removed from the wire filament 30.

In some cases, the elongate tube 26, the wire filament 30, the steering wires 56, and/or other components of the accessory medical device 14 may be configured to return to a relaxed state when forces causing movement of the distal portion 26b of the elongate tube 26 are removed from the handle 28. For example, the elongate tube 26, the wire filament 30, and/or the steering wires 56 may be formed from resilient polymer, metal, and/or combinations of polymer and metal materials that facilitate the wire filament 30 and/or the steering wires 56 returning to the relaxed state.

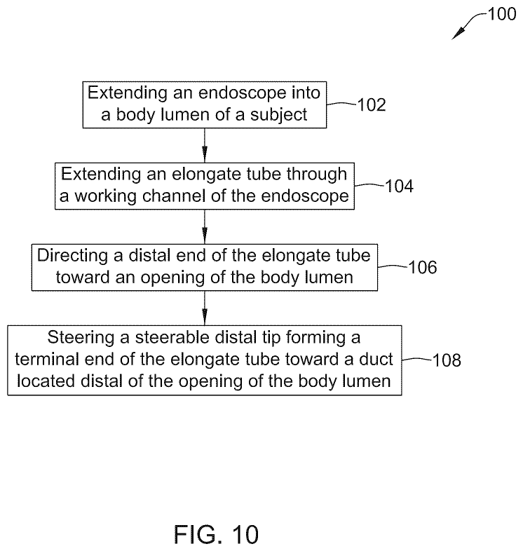

FIG. 10 is a schematic flow diagram of an illustrative technique 100 for accessing a body lumen of a subject. In some cases, a system similar to system 24 discussed above may be utilized for accessing the body lumen of the subject. Although the technique 100 may be utilized for accessing openings of other body lumens, the technique 100, in one example, may be utilized for accessing a desired one of the common bile duct and the pancreatic duct.

The illustrative technique 100 may include extending 102 a medical device (e.g., the medical device 22, such as an endoscope or duodenoscope, and/or other suitable medical device) through a body lumen (e.g., a duodenum or other suitable body lumen) of a subject. In some cases, the medical device may have a working channel configured to receive one or more accessory medical devices (e.g., the accessory medical device 14, such as a sphincterotome, and/or other suitable accessory medical device). The medical device may be extended through the body lumen such that an opening in the medical device and/or a visualizing lens of the medical device is positioned proximate a target location (e.g., a papilla of Vater or an opening associated therewith). A working channel or lumen of the medical device may be in communication with the opening of the medical device positioned proximate to the target location.

An elongate tube (e.g., the elongate tube 26 and/or other suitable elongate tube) may be extended 104 through the working channel of the medical device. In some cases, the elongate tube may be part of an accessory medical device, such as a sphincterotome or other suitable accessory medical device. The elongate tube may be extended through the working channel in any suitable manner including, but not limited to, by threading the elongate tube through the working channel using a rapid exchange technique, over the wire technique, etc.

Further, the technique 100 may include directing 106 a distal end of the elongate tube toward and/or through an opening of the medical device that is in communication with the working channel to the opening of a target body lumen. The distal end of the elongate tube may be directed toward the opening of the target body lumen using any suitable technique. In one example, a ramp or other suitable elevator at a distal end of the working channel of the medical device may direct a distally advancing elongate tube toward and out of the opening in communication with the working channel and toward the opening of the target body lumen. Additionally or alternatively, in another example, a tension may be applied to a wire filament (e.g., the wire filament 30 and/or other suitable wire filament) to cause the distal end of the elongate tube of the accessory medical device to bend in a direction of the wire filament and toward the opening of the body lumen. Tension may be applied to the wire filament in any suitable manner, as discussed herein (e.g., by adjusting an actuating portion in a proximal longitudinal direction) or otherwise.

Once a distal tip (e.g., the distal tip 31 and/or other suitable distal tip) of the elongate tube has been positioned within the opening of the body lumen or to facilitate positioning the distal tip in a target body lumen, the steerable distal tip may be steered 108 toward a duct located distal of the opening of the body lumen. In one example, when the distal tip has been positioned in the opening at the papilla of Vater, the distal tip may be manipulated or controlled so as to be directed to a common bile duct or a pancreatic duct distal of an opening of the papilla by radially manipulating or adjusting a manipulating portion (e.g., the manipulating portion 58 or other suitable manipulating portion of a handle of an accessory medical device) to position the distal tip in a desired direction relative to a target lumen.

Once the distal tip has been steered toward a duct distal of an opening of the body lumen of the subject, a guidewire or other medical device may be passed through the lumen of the elongate tube having an opening in (e.g., in a terminal end of) the distal tip and placed in a target duct or lumen. Then, the accessory medical device may be removed from the subject and one or more other medical devices may be advanced over the placed guidewire to facilitate performing one or more procedures on the subject in the target duct.

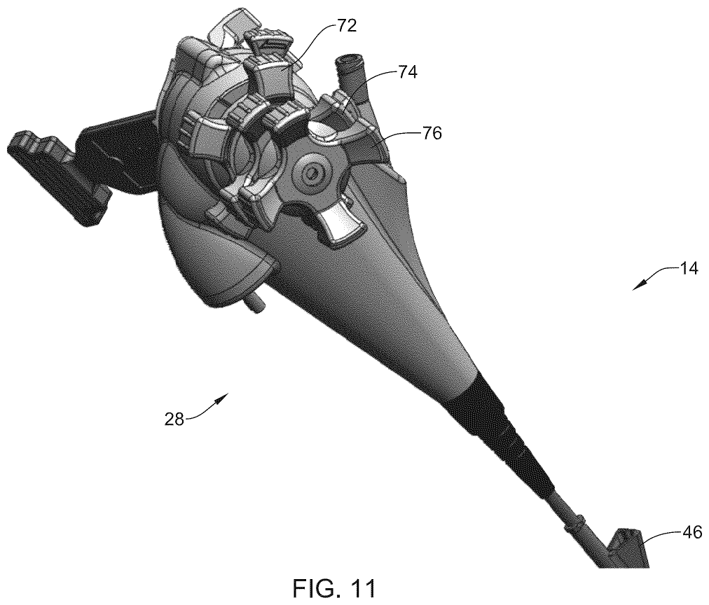

Although manipulation or steering of the distal tip 31 of the accessory medical device 14 has been described herein with respect to actuating or manipulating a second portion 28b of a handle 28 relative to a first portion 28a of the handle, other suitable handle configurations configured to steer or position the distal tip 31 are contemplated. In one example, as schematically depicted in FIG. 11, the handle 28 of the accessory medical device 14 may utilize one or more dials or knobs to adjust the distal tip.

As depicted in FIG. 11, the handle 28 may include a first knob 72, a second knob 74, and a third knob 76. Although FIG. 11 depicts the first knob 72, the second knob 74, and the third knob 76 rotating about a common axis, this is not required and other configurations are contemplated.

Although not required, the first knob 72 and the second knob 74 may each be coupled to two wires in communication with the distal tip 31 (or each coupled to one continuous wire with both ends of the continuous wire anchored at the distal tip 31). When so configured, each knob 72, 74 may be configured to control movement of or otherwise steer the distal tip 31 in two opposing directions. For example, rotation of the first knob 72 in a first rotational direction may result in radial movement of the distal tip 31 in a first radial direction, rotation of the first knob in a second rotational direction opposing the first rotational direction may result in radial movement of the distal tip 31 in a second radial direction opposing the first radial direction, rotation of the second knob 74 in the first rotational direction may result in radial movement of the distal tip 31 in a third radial direction perpendicular to the first and second radial directions, and rotation of the second knob 74 in the second rotational direction may result in radial movement of the distal tip 31 in a fourth radial direction perpendicular to the first and second radial directions and opposite the third radial direction. Adjustment of the first and second knobs 72, 74 in combination with one another may facilitate fine positioning or steering of the distal tip in many directions (potentially any desired direction through a combination of adjustments).

The third knob 76 may be coupled to a proximal end of the wire filament 30. Rotation of the third knob 76 in the first rotational direction may result in applying tension on the wire filament 30 and bending of the distal end 26b of the elongate tube 26, as discussed herein and/or otherwise. Rotation of the third knob 76 in the second rotational direction may result in releasing tension on the wire filament 30 and relaxing of the distal end 26b of the elongate tube 26. Other suitable configurations of the third knob 76 relative to the wire filament 30 are contemplated.

The materials that can be used for the various components of the systems presently disclosed may include those commonly associated with medical devices. For simplicity purposes, the following discussion makes reference to system 24 referenced above. However, this is not intended to limit the devices and methods described herein, as the discussion may be applied to other similar devices and/or components of devices disclosed herein.

The system 24 and/or components thereof may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304 V, 304 L, and 316 LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

Some examples of suitable polymers may include, but are not limited to, polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, a degree of Magnetic Resonance Imaging (MRI) compatibility is imparted into the system 24. For example, the system 24 or portions thereof may be made of a material that does not substantially distort the image and create substantial artifacts (i.e., gaps in the image). Certain ferromagnetic materials, for example, may not be suitable because they may create artifacts in an MM image. The system 24 or portions thereof may also be made from a material that the MM machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

As alluded to above, the tubular and/or elongated components of the system 24 may include one or more tubular members that may have slots formed therein. Various embodiments of arrangements and configurations of slots are contemplated. For example, in some embodiments, at least some, if not all of the slots are disposed at the same or a similar angle with respect to the longitudinal axis of the tubular and/or elongated components of the system 24. The slots can be disposed at an angle that is perpendicular, or substantially perpendicular, and/or can be characterized as being disposed in a plane that is normal to the longitudinal axis of the tubular and/or elongated components of the system 24. However, in other embodiments, the slots can be disposed at an angle that is not perpendicular, and/or can be characterized as being disposed in a plane that is not normal to the longitudinal axis of the tubular components of the system 24. Additionally, a group of one or more the slots may be disposed at different angles relative to another group of one or more the slots. The distribution and/or configuration of the slots can also include, to the extent applicable, any of those disclosed in U.S. Pat. No. 7,914,467, the entire disclosure of which is herein incorporated by reference. Some example embodiments of appropriate micromachining methods and other cutting methods, and structures for tubular members including slots and medical devices including tubular members are disclosed in U.S. Pat. Publication Nos. 2003/0069522 and 2004/0181174-A2; and U.S. Pat. Nos. 6,766,720; and 6,579,246, the entire disclosures of which are herein incorporated by reference. Some example embodiments of etching processes are described in U.S. Pat. No. 5,106,455, the entire disclosure of which is herein incorporated by reference. It should be noted that the methods for manufacturing the system 24 may include forming the slots in the tubular or elongated components of the system 24 using these or other manufacturing steps.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A medical device comprising:
an elongate tube having a proximal end and a distal end configured to be directed toward an opening of a body lumen;
a wire filament extending along the elongate tube, a distal end of the wire filament connected to the distal end of the elongate tube, a proximal portion of the wire filament extending at least partially along the elongate tube, and a distal portion of the wire filament extending external to the elongate tube;
a steerable distal tip extending distal of a distal end of the wire filament; and
a handle in mechanical communication with the steerable distal tip; and
wherein the handle is configured to be adjusted to control a position of the steerable distal tip in at least three directions.

2. The medical device of claim 1, further comprising:
one or more steering wires extending from the handle to the steerable distal tip; and
wherein adjustment of the handle causes tension on one or more of the one or more steering wires to adjust the position of the steerable distal tip.

3. The medical device of claim 2, wherein the handle includes an actuating portion connected to the wire filament and the one or more steering wires.

4. The medical device of claim 3, wherein actuation of the actuating portion in a radial direction adjusts the position of the steerable distal tip.

5. The medical device of claim 3, wherein:
actuation of the actuating portion in an axial direction causes the distal end of the elongate tube to flex; and
actuation of the actuating portion in a radial direction adjusts the radial position of the steerable distal tip.

6. The medical device of claim 3, wherein the actuating portion includes a manipulating element coupled to the one or more steering wires and configured to radially adjust about a rotational joint.

7. The medical device of claim 2, wherein:
the one or more of the steering wires extend through at least a portion of the elongate tube; and
the elongate tube includes a first lumen for receiving the wire filament and one or more lumens each configured to receive one of the one or more steering wires.

8. The medical device of claim 2, wherein the one or more steering wires include at least three steering wires extending from the handle to the steerable distal tip.

9. The medical device of claim 1, wherein adjustment of the handle in a radial direction relative to the elongate tube is configured to adjust the position of the steerable distal tip.

10. The medical device of claim 1, wherein the handle is in communication with the proximal portion of the wire filament and adjustment of the handle in an axial direction of the elongate tube is configured to cause the distal end of the elongate tube to flex and expose a cutting portion of the wire filament.

11. A system, comprising:
an endoscope having a proximal end with an axial opening, a distal end with a radial opening and a working channel extending therebetween;
a guidewire; and
a medical device extendable through the working channel and configured to be directed toward an opening of a body lumen comprising:
an elongate tube having a proximal end and a distal end, the distal end configured to extend through the radial opening;
a steerable distal tip configured to extend through the radial opening;
a handle in mechanical communication with the steerable distal tip; and
wherein the handle is configured to be adjusted to adjust a radial position of the steerable distal tip.

12. The system of claim 11, further comprising:
one or more steering wires extending from the handle to the steerable distal tip; and
wherein adjustment of the handle causes tension on one or more of the steering wires to adjust a radial position of the steerable distal tip.

13. The system of claim 12, wherein:
the handle includes an actuating portion connected to the one or more steering wires; and
actuation of the actuating portion in a radial direction adjusts a radial position of the steerable distal tip.

14. The system of claim 13, wherein the actuating portion includes a manipulating element coupled to the one or more steering wires and configured to radially adjust about a rotational joint.

15. The system of claim 11, wherein the medical device further comprises a wire filament extending along the elongate tube, a distal end of the wire filament is connected to the distal end of the elongate tube, a proximal portion of the wire filament extends at least partially along the elongate tube, and a distal portion of the wire filament extends external to the elongate tube.

16. A method of accessing a body lumen of a subject, comprising:
extending an endoscope having a working channel into the body lumen of the subject;
extending an elongate tube having a distal end and a lumen through the working channel;
directing the distal end of the elongate tube through an opening of the body lumen; and
steering a steerable distal tip forming a terminal end of the elongate tube toward a duct located distal of the opening of the body lumen.

17. The method of claim 16, wherein steering the steerable distal tip comprises radially adjusting a handle in communication with the steerable distal tip to steer the steerable distal tip toward the duct.

18. The method of claim 16, wherein steering the steerable distal tip comprises radially adjusting at least a portion of a handle in communication with the steerable distal tip about a rotational joint to steer the steerable distal tip toward the duct.

19. The method of claim 16, further comprising:
extending a guidewire through the steerable distal tip and into the duct.

20. The method of claim 19, further comprising:
removing the elongate tube from the body lumen and the working channel of the endoscope while maintaining the guidewire in the duct; and
inserting another elongate tube over the guidewire and into the duct.
